(19) United States
(12) Patent Application Publication
(10) Pub. No.: US 2004/0167203 A1
(43) Pub. Date: Aug. 26, 2004

(54) Absorption enhancing agents

(76) Inventors: Rong-Kun Chang, Rockville, MD (US); Benjamin Kibalo, Monmouth Junction, NJ (US); Richard A. Couch, Chevy Chase, MD (US); Mark J. Ginski, Frederick, MD (US); Ali Keshavarz-Shokri, Rockville, MD (US); Caren C. Bancroft, Germantown, MD (US)

Correspondence Address: M. ELISA LANE, SHIRE LABORATORIES INC., 1550 EAST GUDE DRIVE, ROCKVILLE, MD, 20850, US

(21) Appl. No.: 10/762,446

(22) Filed: Jan. 22, 2004

Related U.S. Application Data
(60) Provisional application No. 60/441,950, filed on Jan. 23, 2003.

(51) Int. Cl. A61K 31/385; A61K 31/198; A61K 31/19

(52) U.S. Cl. 514/440; 514/561; 514/557

(57) ABSTRACT

Disclosed are new compounds that increase the absorption of pharmaceutical agents across mucous membranes. These absorption enhancers allow higher bioavailability of administered drugs. The enhancers advantageously have low or no cytotoxicity.

This nonprovisional application claims the benefit of U.S. Provisional Application No. 60/441,950, filed Jan. 22, 2003, the entirety of which is incorporated herein by reference.

### FIELD OF THE INVENTION

The present invention is directed to pharmaceutical compositions that contain, or are administered together with, certain mucosal membrane absorption enhancing compounds. The compositions beneficially increase the bioavailability of the active pharmaceutical agent or agents in the composition.

### BACKGROUND OF THE INVENTION

Many drugs are administered in a manner that requires the therapeutic agent to cross a mucosal membrane cellular layer face factors limiting the bioavailability, and thus the therapeutic performance, of the active agent. For instance, mucosal layers of epithelium are encountered when administering drugs orally, sublingually, buccally, rectally, intranasally, vaginally, and ocularly.

Most systemic drugs are administered enterally, intranasally or by inhalation for patient comfort reasons. “Enterally” for the purposes of this disclosure means any means of administration whereby the drug is absorbed through the gastrointestinal tract, including the oral mucosa. In order for enterally administered drugs to have a systemic affect, they must somehow pass from the lumen of the GI tract to the underlying circulation. The epithelial cells lining the GI tract present a barrier to the efficient absorption of enterally administered drugs. Similarly, the epithelial cells forming the lining of the respiratory system are an obstacle to the efficient absorption of intranasal or inhaled administration. Drug compositions that have the ability to enhance the transport of drugs across the mucosal membranes of various body cavities would be an improvement in the pharmaceutical arts.

It has been found that when poorly absorbed drugs are administered orally or rectally, for instance, the bioavailability of the drugs could be increased by administering them together with absorption enhancer(s). However, most of these enhancers, e.g., sodium salicylate, 5-methoxysalicylate, sodium cholate, S-nitroso-N-acetyl-DL-penicillamine, sodium benzoate, sodium gentisate, sodium lauryl sulfate, etc., can damage and irritate the intestinal mucosal membrane. Therefore, there remains a need in the field for effective, but safe, absorption enhancers.

### SUMMARY OF THE INVENTION

In one aspect, the present invention is directed to a composition comprising at least one pharmaceutically active agent and one or more of N,N-dimethylglycine, thioctic acid, sebacic acid, and shikimic acid, and salts thereof, and methods of preparing the same. The N,N-dimethylglycine, thioctic acid, sebacic acid, and shikimic acid, and salts thereof, act as absorption enhancers for drugs encountering an epithelial barrier, for instance in the gastrointestinal tract, leading to higher bioavailability of the drug. Moreover, these enhancers have low cytotoxicity.

A further aspect of the present invention is a method for enhancing the absorption of a pharmaceutically active agent or agents through mucous membranes of body cavities, comprising administering to the body cavity a combination comprising at least one active agent and one or more of N,N-dimethyl glycine, thioctic acid, sebacic acid, shikimic acid, and salts thereof.

### DETAILED DESCRIPTION OF THE INVENTION

With the present invention it was found that N,N-dimethylglycine, thioctic acid, sebacic acid, and shikimic acid consistently improved the permeability of sampatrilat and hydrochlorothiazide across a Caco-2 cell line that forms a confluent epithelial layer. In addition, these new excipients have low cytotoxicity.

The Caco-2 cell line is a well-recognized in vitro screening model, which both structurally and functionally represents the small intestinal epithelium. Caco-2 cells are derived from human colon carcinoma cells and differentiate in culture to form intestinal epithelia similar to that found in the small intestine. More specifically, Caco-2 cells form a brush border with normal enzymes, form tight junctions between cells, and acquire the barrier properties of an enterocyte sheet. This cell line was utilized to evaluate the absorption enhancers and drug formulations of the present invention in a manner known and which is generally disclosed, for example, in Drug Absorption Enhancement, A. (Bert) G. de Boer, ed., ISBN 3-7186-5492-X (1994), which is incorporated herein by reference, particularly Chapter 3 thereof. The absorption enhancers were tested using two model drugs, sampatrilat and hydrochlorothiazide. In addition, a lactate dehydrogenase (LDH) assay was conducted after the permeation studies to evaluate the cytotoxicity of the absorption enhancers as well as to discover any violation of the integrity of the Caco-2 cells. LDH is a cytosolic enzyme that is not normally secreted outside the cell. However, it leaks into the culture medium upon damage to the cell membranes. In vitro release of LDH from cells provides an accurate measure of cell membrane integrity and cell viability. Although used immunological studies and in studies that test the biocompatibility of implantable biomaterials, the present inventors have found that it is a reliable and accurate test of the cellular toxicity of pharmaceutical excipients such as the enhancers of this invention. Wu, S.-J., et al, Pharmaceutical Res., 16(8): 1266-1272 (1999); Allen, M. J. et al., Promega Notes Magazine, Number 45, p. 7 (1994); or Ehrlich, M. et al, Current Protocols in Toxicology, John Wiley & Sons, New York (2000). LDH leakage into the apical compartment of the Caco-2 cell system was used to measure the effect, if any, of a given absorption enhancer on the viability of the Caco-2 cells.

Compositions according to the present invention are comprised of one or more pharmaceutically active agents, and one or more of the enhancer excipients, N,N-dimethylglycine, thioctic acid, sebacic acid, shikimic acid, and salts thereof. The active agent(s) is one whose bioavailability is increased in the presence of at least one of N,N-dimethylglycine, thioctic acid, sebacic acid, shikimic acid, and/or salts thereof; in other words, any drug, either now known or later discovered, that could benefit from enhanced absorption is advantageously formulated with the enhancers of the present invention. Typically, it would be a drug that exhibits poor bioavailability due to poor permeation of an mucosal epithelial cell layer, such as in the gastrointestinal tract, which would include inter alia such active agents as peptides, proteins and nucleic acids. The present compositions are not limited to a particular drug or combination of drugs, and it is contemplated that the enhancers have widespread applicability. For purposes of demonstration herein there are disclosed formulations of the enhancers with two drugs known for their poor bioavailability, sampatrilat and hydrochlorothiazide, but the invention should not be considered as limited to these exemplary embodiments. In fact, the inventors have found that these two drugs are useful for screening additional absorption enhancer excipients. The amount of pharmaceutically active agent is the typical therapeutic dose, but it is anticipated that a smaller dose will be required because of the enhanced bioavailability.

The compositions of the present invention can contain just one of the enhancers, or a combination of two or more. In general, the enhancers are present in an amount effective to act as an absorption enhancer of the administered drug or drugs, and this amount can be estimated empirically. An amount effective can be one that increases the bioavailability of the drug to any appreciable extent. The enhancers can be present in a concentration in the final dosage form of from about 0.01 % to about 99% by weight, alone or in combination. Preferably, the enhancers are present in the final composition at about 0.01% to about 50% by weight, and more preferably about 0.1% to about 30% by weight. The optimal amount in a given formulation can, of course, be estimated or determined by experimentation such as that described in the examples.

The compositions are in a form suitable for oral, nasal, buccal, sublingual, topical, rectal, or vaginal administration, and may be in the form of liquids, solids, lotions, gels, aerosols, or any other pharmaceutical vehicle. For oral administration, the compositions may be in the form of liquids, suspensions, emulsions, powders, pills, tablets, capsules, gel caps, troches, cachets, pellets, and the like. With pharmaceutically suitable liquids the compositions can take the form of a solution, suspension (or dispersions), aerosol or emulsion, which can be sprayed or inhaled.

The formulations may be prepared by any methods well known in the art of pharmacy, for example, using methods such as those described in Gennaro et al., Remington's Pharmaceutical Sciences (18th ed., Mack Publishing Company, 1990, see especially Part 8: Pharmaceutical Preparations and their Manufacture). Such methods comprise the step of bringing into association the drug(s), pharmaceutical carrier and enhancer(s). Prior to admixing with the pharmaceutical agent and accessory ingredients (if desired), the enhancer may be solubilized in an appropriate solvent system, such that the final concentration of enhancer(s) in the compositions of the present invention is between about 0.01% to about 99% by weight, preferably about 0.1% and about 50% by weight, and more preferably between about 0.1% and about 30% by weight. Pharmaceutical carriers are suitable vehicles in which the drug or drugs (or “pharmaceutically active agent”) are incorporated in by dissolving, dispersing, or suspending, and include such vehicles as, for example, solvents, lipids, proteins, carbohydrates, polymers, etc., and substances that are added to increase solubility or dispersion of the active agent, such as solubilizers, emulsifiers, and surfactants, for instance. Other accessory ingredients include those conventional in the art, such as fillers, binders, diluents, disintegrants, glidants, lubricants, colorants, flavoring agents and wetting agents.

As preferred embodiments are those compositions that are administered orally and which increase the absorption of the active ingredient(s) in the gastrointestinal tract. For oral administration, the compositions may be in the form of liquids, suspensions, emulsions, powders, pills, tablets, capsules, troches, cachets, pellets, effervescent powders or granules, gel caps, and the like. These dosage forms are prepared in manners known in the art, such as disclosed in Gennaro et al., Remington's Pharmaceutical Sciences, supra.

A further aspect of the present invention is a method for enhancing the absorption across a mucosal membrane of a pharmaceutically active agent, which comprises administering a composition comprising the active agent (or agents) and one or more of N,N-dimethylglycine, thioctic acid, sebacic acid, shikimic acid, and or their salts.

Another embodiment of the present invention is a method for testing the potency of an absorption enhancer in vitro. To practice this aspect of the invention, a confluent monolayer of Caco-2 cells is grown on a permeable support in a culture chamber with apical and basolateral sides. Then, a drug selected from sampatrilat or hydrochlorothiazide is added concurrently or sequentially with a potential enhancer compound to the apical side of the chamber, and after a predetermined time, the amount of drug that passes from the apical side to the basolateral side of the chamber is measured (for instance by transepithelial electrical resistance). The potency of the enhancer is measured by comparing the measurement obtained with the enhancer with a measurement obtained from the addition of drug alone to the apical side of the chamber. The magnitude of any increase is an indication of the potency of the enhancer.

The use of the permeation enhancers of the invention to promote mucosal membrane absorption affords several advantages over the prior art's non-related absorption promoting compounds. The permeation enhancers of the invention are more potent than the currently available absorption promoting agents. As an example, at 1 % w/v concentration, thioctic acid can effectively enhance hydrochlorothiazide permeability across a Caco-2 monolayer 13-fold more than the patented permeation enhancer, 18 β-glycyrrhetinic acid. This difference in potency allows opportunities for reducing the required size of the dosage form and potentially minimizing side effects. Additionally, the results from the lactose dehydrogenase assay reveal that the enhancers (i.e., N,N-dimethyl glycine, thioctic acid, sebacic acid, shikimic acid) are not cytotoxic relative to cells treated with Hank's balanced salt solution alone.

### EXAMPLES

### Example 1

Sampatrilat is a hydrophilic compound containing one weakly acidic phenolic group, two more strongly acidic carboxylic acid groups, and one strong basic primary amine group with an aqueous solubility of 1.8 mg/mL. The compound has relatively low oral bioavailability, primarily due to its poor intestinal permeability. Earlier studies demonstrated about 2 -5% oral bioavailability in vivo when administered by a tablet dosage form. Thus, sampatrilat is a good low permeability model drug.

In this example and Example 2, Caco-2 cells were grown to confluence on permeable supports mounted in a chamber that has an apical side and a basolateral side. Sampatrilat and enhancer were added to the apical chamber to give a concentration of 1.8 mg/mL and 1% w/v, respectively. Permeability coefficients are determined as previously reported by Yazdania et.el (Yazdanian M, Glynn, S I, Wright J L, et al. 1998. Correlating partitioning and Caco-2 permeability of structurally diverse small molecular weight compounds. Pharm Res 15:1490-1494). Briefly, drug solutions were prepared in HBSS at a known final concentration. For AP to BL experiments, the solution was placed on the apical side of the cells and samples were taken from basolateral side. In contrast, for BL to AP experiments, the solution was placed on the basolateral side of the cells and samples were taken from apical side. The samples are analyzed by an HPLC. Transport rates (J) are determined by plotting cumulative amounts of drug permeated as a function of time. Apparent permeability coefficients P_{caco-}2, are determined according to the equation P_{caco-}2=J/ACi where Ci is the initial concentration of the solution in donor chamber and A is the surface area of the filter.

Table 1 shows the calculated permeability coefficients from the Caco-2 transport study. N,N-dimethylglycine, thioctic acid, sebacic acid, and shikimic acid significantly increase the sampatrilat permeation across the Caco-2 cell line. As an example, N,N-dimethylglycine increases sampatrilat permeability 124-fold over the drug alone. The original cell line integrity and the effect of excipients on the integrity of cell line were also tested by measuring the flux of ¹⁴C-mannitol. Except for thioctic acid, it is clear from the data that markedly enhanced transport of sampatrilat by N,N-dimethylglycine, sebacic acid, and shikimic acid coincided with the increased transport of mannitol. Although not intending to be bound to any particular theory, the parallel-enhanced transport of mannitol may indicate that N,N-dimethylglycine, sebacic acid, and shikimic acid increases the paracellular permeation of sampatrilat by opening the tight junctions within the epithelial barrier.

TABLE 1
Permeability coefficients of sampatrilat transport
across Caco-2 cell line
Permeability Coefficient, 10E−7 cm/s

| | | |
|---|---|---|
| Compound | Sampatrilat | Mannitol |

| | | | |
|---|---|---|---|
| Control | No drug | N/A | 46 |
| PD0058- | Drug alone¹ | 1.6 | 4.9 |
| 152A | | | |
| PD0058- | Sebacic | 21.0 | 75.0 |
| 152B | acid² | | |
| PD0058- | Amino | 4.8 | 10.7 |
| 152C | caproic acid² | | |
| PD0058- | N, n- | 199.0 | 123.0 |
| 152D | dimethyl | | |
| | glycine² | | |
| PD0058- | Thioctic | 166.0 | 6.0 |
| 152E | acid² | | |
| PD0058- | Citrulline² | 3.4 | 2.2 |
| 152F | | | |
| PD0058- | Kojic acid² | 4.9 | 16.3 |
| 152G | | | |
| PD0058- | Shikimic | 36.5 | 158.0 |
| 152H | acid² | | |

¹Sampatrilat concentration at 1.8 mg/mL was used for all the Caco-2 transport studies.
²The concentration at 1% w/v was used for all the excipients in this Caco-2 study.

### Example 2

Hydrochlorothiazide is another known low permeability compound. Again, N,N-dimethylglycine, thioctic acid, sebacic acid, and shikimic acid were demonstrated as permeability enhancers in the Caco-2 transport studies using hydrochlorothiazide as a model drug (Table 2). Additionally, the results from the lactose dehydrogenase assay reveal that the excipients (i.e., N,N-dimethylglycine, thioctic acid, sebacic acid, shikimic acid) are not cytotoxic relative to cells treated with Hank's balanced salt solution alone.

Several patented absorption-promoting agents (e.g., cyclopentadecanolide, U.S. Pat. Nos. 5731303 and 5023252; glycyrrhetinic acid, U.S. Pat. No. 6214378; piperine, U.S. Pat. No. 5616593; and Vitamin E TPGS, U.S. Pat. Nos. 5891845 and 5234695) were examined for their permeability enhancing effect and are also shown in Tale 2. As can be seen, these agents show low or no potency in permeability enhancement, compared to the agents of the present invention.

TABLE 2
Permeability coefficients of hydrochlorothiazide transport
across Caco-2 cell line

| | | | Permeability |
|---|---|---|---|
| | | Study | coefficient, |
| Lot number | Sample description | number | 10E−7, cm/s |
| PD0058-161A | N,N-dimethylglycine | 1 | 242 |
| PD0058-161B | Thioctic acid | 1 | 252 |
| PD0058-161C | Cyclopentadecanolide | 1 | 20.3 |
| PD0058-161D | Drug alone | 1 | 25.3 |
| PD0058-161E | Glycyrrehetinic acid | 1 | 19.3 |
| PD0058-166C | Thioctic acid | 2 | 375 |
| PD0058-166E | Piperine | 2 | 14.2 |
| PD0058-166F | Drug alone | 2 | 16.7 |
| PD0058-166G | N,N-dimethylglycine | 2 | 347 |
| PD0058-167D | Sebacic acid | 3 | 73.0 |
| PD0058-167E | Shikimic acid | 3 | 135 |
| PD0058-167F | Vitamin E TGPS | 3 | 5.78 |
| PD0058-167H | Drug alone | 3 | 5.97 |
| PD0058-168B | Drug alone | 4 | 4.69 |
| PD0058-168F | N,N-dimethylglycine | 4 | 344 |
| PD0058-169A | Drug alone | 5 | 4.40 |
| PD0058-169E | Piperine¹ | 5 | 4.34 |
| PD0058-169G | Shikimic acid² | 5 | 364 |
| PD0058-169H | Cyclopentadecanolide¹ | 5 | 6.87 |

Note:
Hydrochlorothiazide concentration at 0.2 mg/mL was used for all the experiments. Excipient concentration at 1% was used for the study #1 to #4. For the study #5, higher excipient concentration was tested.
¹5% w/v concentration
²3% w/v concentration

What is claimed is:

1. A composition comprising at least one pharmaceutically active agent and an enhancer containing one or more of N,N-dimethylglycine, thioctic acid, sebacic acid, shikimic acid, and salts thereof.

2. The composition of claim 1, wherein the active agent is one whose absorption is increased in the presence of said enhancer.

3. The composition of claim 1, wherein the enhancer is present in the composition in an amount effective to increase the biological absorption of the active agent.

4. The composition of claim 3, wherein the concentration of the enhancer is from about 0.01% to about 99% by weight.

5. The composition of claim 4, wherein the concentration of enhancer is from about 0.01% to about 50% by weight.

6. The composition of claim 4, wherein the concentration of enhancer is from about 0.1% to about 30% by weight.

7. The composition of claim 1, which comprises at least one pharmaceutically active agent and N,N-dimethylglycine or its salt.

8. The composition of claim 1, which comprises at least one pharmaceutically active agent and thioctic acid or its salt.

9. The composition of claim 1, which comprises at least one pharmaceutically active agent and sebacic acid or its salt.

10. The composition of claim 1, which comprises at least one pharmaceutically active agent and thioctic acid or its salt.

11. The composition of claim 1, wherein the active agent is a protein, peptide, or nucleic acid.

12. The composition of claim 1, wherein the active agent is selected from sampatrilat and hydrochlorothiazide.

13. The composition of claim 1, which is an oral pharmaceutical in the form of a liquid, suspension, emulsion, powder, pill, tablet, capsule, gel caps, troche, cachet or pellet.

14. The composition of claim 1, which is in the form of a solution, suspension, aerosol, or emulsion, which can be sprayed or inhaled.

15. A method for enhancing the absorption of a pharmaceutically active agent across a mucosal membrane in a mammal, comprising administering to the mammal a composition comprising at least one active agent and an enhancer containing one or more of N,N-dimethylglycine, thioctic acid, sebacic acid, shikimic acid, and salts thereof.

16. The method of claim 13, wherein the active agent is one whose bioavailability is increased in the presence of said enhancer.

17. The method of claim 13, wherein the enhancer is present in the composition in an amount effective to increase the bioavailability of the active agent.

18. The method of claim 15, wherein the concentration of the enhancer is from about 0.01 % to about 99% by weight.

19. The method of claim 16, wherein the concentration of the enhancer is from about 0.01% to about 50% by weight.

20. The method of claim 17, wherein the concentration of the enhancer is from about 0.1% to about 30% by weight.

21. The method of claim 13, wherein the mucosal membrane is the gastrointestinal tract and the composition is administered orally, buccally or sublingually.

22. The method of claim 18, wherein the composition is administered orally.

23. A process for preparing the composition of claim 1, comprising bringing into association at least one pharmaceutically active agent with one or more enhancer, and forming a liquid, suspension, emulsion, aerosol, powder, pill, tablet, capsule, gel caps, troche, cachet or pellet therewith.

24. The process of claim 23, which further comprises adding a pharmaceutical carrier or carrier to the active agent and enhancer.

25. A process for testing the potency of an absorption enhancer in vitro, comprising:
(a) growing a confluent monolayer of Caco-2 cells on a permeable support in a culture chamber with apical and basolateral sides;
(b) adding a drug selected from sampatrilat or hydrochlorothiazide concurrently or sequentially with a potential enhancer compound to the apical side of the chamber;
(c) after a predetermined time measuring the amount of drug that passes from the apical side to the basolateral side of the chamber; and
(d) comparing the measurement from step (c) with a measurement obtained from the addition of drug alone to the apical side of the chamber,
whereby the magnitude of any increase is an indication of the potency of the enhancer.
